(19) United States
(12) Patent Application Publication
(10) Pub. No.: US 2013/0059896 A1
(43) Pub. Date: Mar. 7, 2013

(54) 5-(1H-PYRAZOL-5-YL)THIAZOLE-BASED COMPOUNDS FOR THE TREATMENT OF DISEASES AND DISORDERS OF THE EYE

(71) Applicants: Nicole Cathleen Goodwin, Pennington, NJ (US); David Brent Rawlins, Morrisville, PA (US)

(72) Inventors: Nicole Cathleen Goodwin, Pennington, NJ (US); David Brent Rawlins, Morrisville, PA (US)

(21) Appl. No.: 13/574,024

(22) PCT Filed: Jan. 21, 2011
(86) PCT No.: PCT/US2011/021970

§ 371 Date: Sep. 14, 2012

Related U.S. Application Data
(60) Provisional application No. 61/297,397, filed on Jan. 22, 2010.

(51) Int. Cl. C07D 417/04; A61P 27/06; A61P 27/02; A61K 31/427; C07D 417/14

(52) U.S. Cl. 514/371; 548/195

(57) ABSTRACT

Inhibitors of LIM kinase 2 are disclosed, along with pharmaceutical compositions comprising them and methods of their use. Particular compounds are of the formula (I)

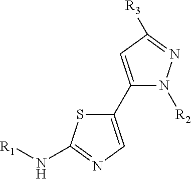

### 1. FIELD OF THE INVENTION

This invention relates to kinase inhibitors, compositions comprising them, and methods of their use to treat various diseases and disorders.

### 2. BACKGROUND

Protein kinases are a class of enzymes that catalyze the transfer of the γ-phosphate group from ATP to a recipient protein. The human genome is estimated to encode in excess of 500 distinct protein kinases, of which many have been implicated in a wide range of diseases and disorders, including cancer and inflammation.

The LIM kinases (LIMK) have been linked to the p53 pathway. See, e.g., International Application No. WO 02/099048. LIMK belongs to a small subfamily of kinases with a unique combination of two N-terminal LIM motifs and a C-terminal protein kinase domain. These LIM motifs and kinase domains are linked by a proline- and serine-rich region containing several putative casein kinase and map kinase recognition sites. LIM kinases and their pathway proteins are believed to contribute to Rho-induced reorganization of the actin cytoskeleton. Id. Members of the LIM kinase family include LIM kinase 1 (LIMK1) and LIM kinase 2 (LIMK2). Both phosphorylate cofilin and regulates Rho family-dependent actin cytoskeletal rearrangement. Id.

LIM kinase inhibitors have been proposed for the treatment of cancer. Id.; International Application No. WO 2003003016 ; Stanyon, Clement A. and Bernard, Ora., Int. J. Biochem. & Cell Biol. 31(3/4): 389-394 (1999); Yoshioka, Kiyoko et al., Proc. National Acad. Sci. USA 100(12): 7247-7252 (2003). It has also been suggested that LIMK inhibitors may be useful in treating glaucoma by promoting actin depolymerization in trabecular cells and lowering ocular tension. See International Application No. WO 04/047868. See also U.S. patent application publication nos. US-2009-0042893-A1 and US-2009-0264450-A1. Current glaucoma therapies operate by different mechanisms. Prostaglandin F2a analogues (e.g., latanoprost) effect an intraocular pressure (IOP) independent increase in fluid outflow from the eye. Carbonic anhydrous inhibitors (e.g., acetazolamide), beta-blockers (e.g., timolol), sympathomimetics (e.g., pilocarpine), and alpha adrenergic receptor agonists (e.g., brimonidine) decrease aqueous humor production.

### 3. SUMMARY OF THE INVENTION

This invention is directed, in part, to compounds of the formula:

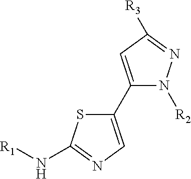

and pharmaceutically acceptable salts thereof, the substituents of which are defined herein. Particular compounds are potent inhibitors of LIMK2.

One embodiment of the invention encompasses pharmaceutical formations comprising compounds disclosed herein.

Another embodiment encompasses methods of using the compounds disclosed herein for the treatment, management and prevention of various diseases and disorders affecting vision (e.g., diseases and disorders of the eye), such as glaucoma, neurodegeneration and infection.

### 4. BRIEF DESCRIPTION OF THE FIGURE

FIG. 1 shows the dose response of a compound of the invention, (S)—N-(5-(1-(2,6-dichlorophenyl)-3-(difluoromethyl)-1H-pyrazol-5-yl)thiazol-2-yl)-2-(pyrrolidin-2-yl)acetamide, in the ocular hypertension assay described in the Examples below.

### 5. DETAILED DESCRIPTION

### 5.1. Definitions

Unless otherwise indicated, the term “alkenyl” means a straight chain, branched and/or cyclic hydrocarbon having from 2 to 20 (e.g., 2 to 10 or 2 to 6) carbon atoms, and including at least one carbon-carbon double bond. Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl.

Unless otherwise indicated, the term “alkoxy” means an —O-alkyl group. Examples of alkoxy groups include, but are not limited to, —OCH₃, —OCH₂CH₃, —OCH₂)₂CH₃, —OCH₂)₃CH₃, —O(CH₂)₄CH₃, and —O(CH₂)₅CH₃.

Unless otherwise indicated, the term “alkyl” means a straight chain, branched and/or cyclic (“cycloalkyl”) hydrocarbon having from 1 to 20 (e.g., 1 to 10 or 1 to 4) carbon atoms. Alkyl moieties having from 1 to 4 carbons are referred to as “lower alkyl.” Examples of alkyl groups include, but are not limited to, methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). The term “alkyl” includes saturated hydrocarbons as well as alkenyl and alkynyl moieties.

Unless otherwise indicated, the term “alkylaryl” or “alkyl-aryl” means an alkyl moiety bound to an aryl moiety.

Unless otherwise indicated, the term “alkylheteroaryl” or “alkyl-heteroaryl” means an alkyl moiety bound to a heteroaryl moiety.

Unless otherwise indicated, the term “alkylheterocycle” or “alkyl-heterocycle” means an alkyl moiety bound to a heterocycle moiety.

Unless otherwise indicated, the term “alkynyl” means a straight chain, branched or cyclic hydrocarbon having from 2 to 20 (e.g., 2 to 20 or 2 to 6) carbon atoms, and including at least one carbon-carbon triple bond. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl.

Unless otherwise indicated, the term “aryl” means an aromatic ring or an aromatic or partially aromatic ring system composed of carbon and H atoms. An aryl moiety may comprise multiple rings bound or fused together. Examples of aryl moieties include, but are not limited to, anthracenyl, azulenyl, biphenyl, fluorenyl, indan, indenyl, naphthyl, phenanthrenyl, phenyl, 1,2,3,4-tetrahydro-naphthalene, and tolyl.

Unless otherwise indicated, the term “arylalkyl” or “aryl-alkyl” means an aryl moiety bound to an alkyl moiety.

Unless otherwise indicated, the terms “halogen” and “halo” encompass fluorine, chlorine, bromine, and iodine.

Unless otherwise indicated, the term “heteroalkyl” refers to an alkyl moiety (e.g., linear, branched or cyclic) in which at least one of its carbon atoms has been replaced with a heteroatom (e.g., N, O or S).

Unless otherwise indicated, the term “heteroalkylaryl” or “heteroalkyl-aryl” refers to a heteroalkyl moiety bound to an alkyl moiety.

Unless otherwise indicated, the term “heteroalkylheterocycle” or “heteroalkyl-heterocycle” refers to a heteroalkyl moiety bound to heterocycle moiety.

Unless otherwise indicated, the term “heteroaryl” means an aryl moiety wherein at least one of its carbon atoms has been replaced with a heteroatom (e.g., N, O or S).

Examples include, but are not limited to, acridinyl, benzimidazolyl, benzofuranyl, benzoisothiazolyl, benzoisoxazolyl, benzoquinazolinyl, benzothiazolyl, benzoxazolyl, furyl, imidazolyl, indolyl, isothiazolyl, isoxazolyl, oxadiazolyl, oxazolyl, phthalazinyl, pyrazinyl, pyrazolyl, pyridazinyl, pyridyl, pyrimidinyl, pyrimidyl, pyrrolyl, quinazolinyl, quinolinyl, tetrazolyl, thiazolyl, and triazinyl.

Unless otherwise indicated, the term “heteroarylalkyl” or “heteroaryl-alkyl” means a heteroaryl moiety bound to an alkyl moiety.

Unless otherwise indicated, the term “heterocycle” refers to an aromatic, partially aromatic or non-aromatic monocyclic or polycyclic ring or ring system comprised of carbon, H and at least one heteroatom (e.g., N, O or S). A heterocycle may comprise multiple (i.e., two or more) rings fused or bound together. Heterocycles include heteroaryls. Examples include, but are not limited to, benzo[1,3]dioxolyl, 2,3-dihydro-benzo[1,4]dioxinyl, cinnolinyl, furanyl, hydantoinyl, morpholinyl, oxetanyl, oxiranyl, piperazinyl, piperidinyl, pyrrolidinonyl, pyrrolidinyl, tetrahydrofuranyl, tetrahydropyranyl, tetrahydropyridinyl, tetrahydropyrimidinyl, tetrahydrothiophenyl, tetrahydrothiopyranyl and valerolactamyl.

Unless otherwise indicated, the term “heterocyclealkyl” or “heterocycle-alkyl” refers to a heterocycle moiety bound to an alkyl moiety.

Unless otherwise indicated, the term “heterocycloalkyl” refers to a non-aromatic heterocycle.

Unless otherwise indicated, the term “heterocycloalkylalkyl” or “heterocycloalkyl-alkyl” refers to a heterocycloalkyl moiety bound to an alkyl moiety. Unless otherwise indicated, the term “LIMK2 IC₅₀” is the IC₅₀ of a compound determined using the in vitro human LIM kinase 2 inhibition assay described in the Examples, below.

Unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

Unless otherwise indicated, the term “pharmaceutically acceptable salts” refers to salts prepared from pharmaceutically acceptable non-toxic acids or bases including inorganic acids and bases and organic acids and bases. Suitable pharmaceutically acceptable base addition salts include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art. See, e.g., Remington's Pharmaceutical Sciences, 18^{th} ed. (Mack Publishing, Easton Pa.: 1990) and Remington: The Science and Practice of Pharmacy, 19^{th} ed. (Mack Publishing, Easton Pa.: 1995).

Unless otherwise indicated, a “potent LIMK2 inhibitor” is a compound that has a LIMK2 IC₅₀ of less than about 250 nM. Unless otherwise indicated, the terms “prevent,” “preventing” and “prevention” contemplate an action that occurs before a patient begins to suffer from the specified disease or disorder, which inhibits or reduces the severity of the disease or disorder. In other words, the terms encompass prophylaxis.

Unless otherwise indicated, a “prophylactically effective amount” of a compound is an amount sufficient to prevent a disease or condition, or one or more symptoms associated with the disease or condition, or prevent its recurrence. A “prophylactically effective amount” of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

Unless otherwise indicated, the term “substituted,” when used to describe a chemical structure or moiety, refers to a derivative of that structure or moiety wherein one or more of its H atoms is substituted with a chemical moiety or functional group such as, but not limited to, alcohol, aldehyde, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (e.g. —C(O)NH-alkyl-, -alkylNHC(O)alkyl), amidinyl (e.g., —C(NH)NH-alkyl-, —C(NR)NH₂), amine (primary, secondary and tertiary such as alkylamino, arylamino, arylalkylamino), aroyl, aryl, aryloxy, azo, carbamoyl (e.g., —NHC(O)O-alkyl-, —OC(O)NH-alkyl), carbamyl (e.g., CONH₂, CONH-alkyl, CONH-aryl, CONH-arylalkyl), carbonyl, carboxyl, carboxylic acid, carboxylic acid anhydride, carboxylic acid chloride, cyano, ester, epoxide, ether (e.g., methoxy, ethoxy), guanidino, halo, haloalkyl (e.g., —CCl₃, —CF₃, —C(CF₃)₃), heteroalkyl, hemiacetal, imine (primary and secondary), isocyanate, isothiocyanate, ketone, nitrile, nitro, oxo, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) and urea (e.g., —NHCONH-alkyl-).

Unless otherwise indicated, a “therapeutically effective amount” of a compound is an amount sufficient to provide a therapeutic benefit in the treatment or management of a disease or condition, or to delay or minimize one or more symptoms associated with the disease or condition. A “therapeutically effective amount” of a compound means an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Unless otherwise indicated, the terms “treat,” “treating” and “treatment” contemplate an action that occurs while a patient is suffering from the specified disease or disorder, which reduces the severity of the disease or disorder, or retards or slows the progression of the disease or disorder.

Unless otherwise indicated, the term “include” has the same meaning as “include, but are not limited to,” and the term “includes” has the same meaning as “includes, but is not limited to.” Similarly, the term “such as” has the same meaning as the term “such as, but not limited to.”

Unless otherwise indicated, one or more adjectives immediately preceding a series of nouns is to be construed as applying to each of the nouns. For example, the phrase “optionally substituted alky, aryl, or heteroaryl” has the same meaning as “optionally substituted alky, optionally substituted aryl, or optionally substituted heteroaryl.”

It should be noted that a chemical moiety that forms part of a larger compound may be described herein using a name commonly accorded it when it exists as a single molecule or a name commonly accorded its radical. For example, the terms “pyridine” and “pyridyl” are accorded the same meaning when used to describe a moiety attached to other chemical moieties. Thus, the two phrases “XOH, wherein X is pyridyl” and “XOH, wherein X is pyridine” are accorded the same meaning, and encompass the compounds pyridin-2-ol, pyridin-3-ol and pyridin-4-ol.

It should also be noted that if the stereochemistry of a structure or a portion of a structure is not indicated with, for example, bold or dashed lines, the structure or the portion of the structure is to be interpreted as encompassing all stereoisomers of it. Moreover, any atom shown in a drawing with unsatisfied valences is assumed to be attached to enough H atoms to satisfy the valences. In addition, chemical bonds depicted with one solid line parallel to one dashed line encompass both single and double (e.g., aromatic) bonds, if valences permit.

### 5.2. Compounds

This invention encompasses compounds of the formula:

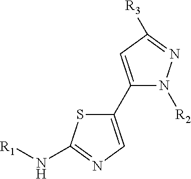

and pharmaceutically acceptables salt thereof, wherein: R₁ is H, C(O)R_{A}, S(O)ₙR_{A}, C(O)NR_{A}R_{B}, S(O)ₙNR_{A}R_{B}, S(O)ₙOR_{A}, C(NH)NR_{A}R_{B}, C(O)OR_{A}, C(S)NR_{A}R_{B}, C(SR_{B})NR_{A}, P(O)(OR_{A})₂or optionally substituted alkyl, aryl, or heterocycle (e.g., optionally substituted with halo, alkyl, alkoxyl, aryl, heteroaryl, hydroxyl, cyano, NR_{A}R_{B}, SR_{A}, P(O)(OR_{A})₂, CO₂R_{A}, C(O)NR_{A}R_{B}, S(O)ₙR_{A}, S(O)NR_{A}R_{B}, or halogenated (e.g., fluorinated) alkyl, aryl or heteroaryl); R₂ is H, C(O)R_{A}, S(O)ₙR_{A}, C(O)NR_{A}R_{B}, S(O)ₙNR_{A}R_{B}, S(O)ₙOR_{A}, or optionally substituted alkyl, aryl, or heterocycle (e.g., optionally substituted with halo, alkyl, alkoxyl, aryl, heteroaryl, hydroxyl, cyano, NR_{A}R_{B}, SR_{A}, P(O)(OR_{A})₂, CO₂R_{A}, C(O)NR_{A}R_{B}, S(O)ₙR_{A}, S(O)NR_{A}R_{B}, or halogenated (e.g., fluorinated) alkyl, aryl or heteroaryl); R₃ is H, halogen, OR, NR_{A}R_{B}, optionally substituted alkyl (e.g., optionally substituted with halo, alkyl, alkoxyl, hydroxyl, cyano, NR_{A}R_{B}, SR_{A}, CO₂R_{A}, C(O)NR_{A}R_{B}; each R_{A} is independently H or optionally substituted alkyl, aryl, alkylaryl, or alkyl-heterocycle (e.g., optionally substituted with halo, alkyl, alkoxyl, aryl, heteroaryl, hydroxyl, cyano, NR_{A}R_{B}, SR_{A}, P(O)(OR_{A})₂, CO₂R_{A}, C(O)NR_{A}R_{B}, S(O)ₙR_{A}, S(O)NR_{A}R_{B}, or halogenated (e.g., fluorinated) alkyl, aryl or heteroaryl); each R_{B} is optionally substituted alkyl or aryl (e.g., optionally substituted with halo, alkyl, alkoxyl, aryl, heteroaryl, hydroxyl, cyano, NR_{A}R_{B}, SR_{A}, P(O)(OR_{A})₂, CO₂R_{A}, C(O)NR_{A}R_{B}, S(O)ₙR_{A}, S(O)NR_{A}R_{B}, or halogenated (e.g., fluorinated) alkyl, aryl or heteroaryl); or when R_{A} and R_{B} are attached to the same nitrogen atom, they can be taken together with that nitrogen atom to form an optionally substituted heterocycle (e.g., piperidinyl, morpholino, thiomorpholino, piperazinyl, pyrrolidino, and azetidino optionally substituted with halo, alkyl, alkoxyl, aryl, heteroaryl, hydroxyl, cyano, NR_{A}R_{B}, SR_{A}, P(O)(OR_{A})₂, CO₂R_{A}, C(O)NR_{A}R_{B}, S(O)ₙR_{A}, S(O)NR_{A}R_{B}, or halogenated (e.g., fluorinated) alkyl, aryl or heteroaryl); and n is 0-2.

In a particular embodiment, the compound is such that one or more of the following are true: when R₁ is C(O)R_{A}, R₂ is CH F₂, and R₃ is 2,6-dichlorophenyl, R_{A} is not ethoxy, cyclopropyl, or isopropyl; when R₁ is C(O)R_{A}, R₂ is H or CHF₂, and R₃ is 3,5-dimethylphenyl, R_{A} is not methoxy; when R₁ is C(O)NR_{A}R_{B}, R₂ is pyrazyl, R₃ is 2,6-dimethyl-4-methoxyphenyl, and R_{A} is H, R_{B} is not ethyl; or when R₁ is H, and R₂ is methyl, R₃ is not chloro.

In one embodiment, the compound is of the formula:

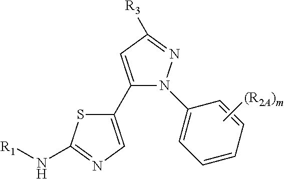

wherein each R_{2A} is independently cyano, halo, hydroxyl, NR_{A}R_{B}, SR_{A}, P(O)(OR_{A})₂, CO₂R_{A}, C(O)NR_{A}R_{B}, S(O)ₙR_{A}, S(O)NR_{A}R_{B}, or optionally substituted (e.g., optionally fluorinated) alkyl, alkoxyl, or aryl; and m is 0-5.

In another, the compound is of the formula:

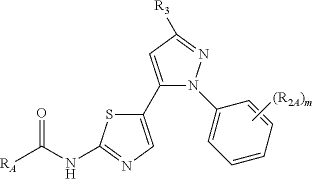

In particular compounds encompassed by formulae described herein, R_{A} is alkyl optionally substituted with one or more of halo, hydroxyl, amino, alkylamino or dialkylamino. In some, R_{A} is isopropyl. In some, R_{A} is alkyl substituted with amino. In some, at least one R_{2A} is chloro.

In one embodiment, the compound is of the formula:

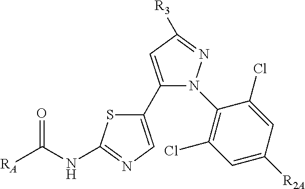

In particular compounds encompassed by formulae described herein, R_{2A} is bromo. In some, m is 2 or 3. In some, R₃ is H or optionally substituted lower alkyl. In some, R₃ is difluoromethyl.

Particular compounds of the invention are potent LIMK2 inhibitors. Certain compounds have a LIMK2 IC₅₀ of less than about 100, 75, 50, 25 or 10 nM.

### 5.3. Methods of Synthesis

Compounds of the invention can be synthesized from common intermediate N-(2,4-dimethoxybenzyl)-4-acetyl-2-aminothiazole (a), which may be prepared by methods known in the art. One approach is described in Ross-MacDonald, et al., Mol. Cancer Ther. 7:3490 (2008), shown below in Scheme 1:

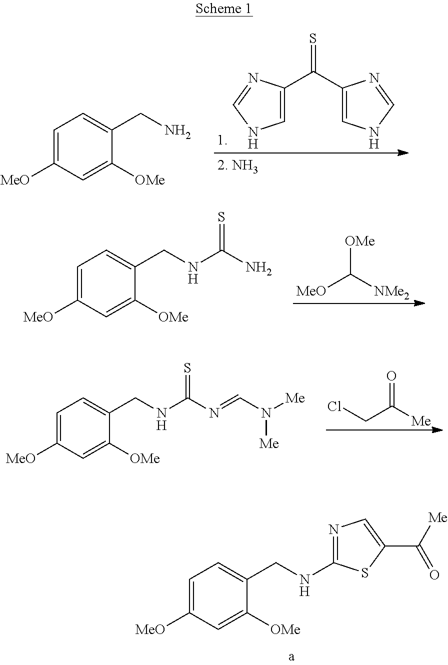

The resulting N-(2,4-dimethoxy)-4-acetyl-2-aminothiazole (a) is then converted to compounds of the invention, as shown below in Scheme 2:

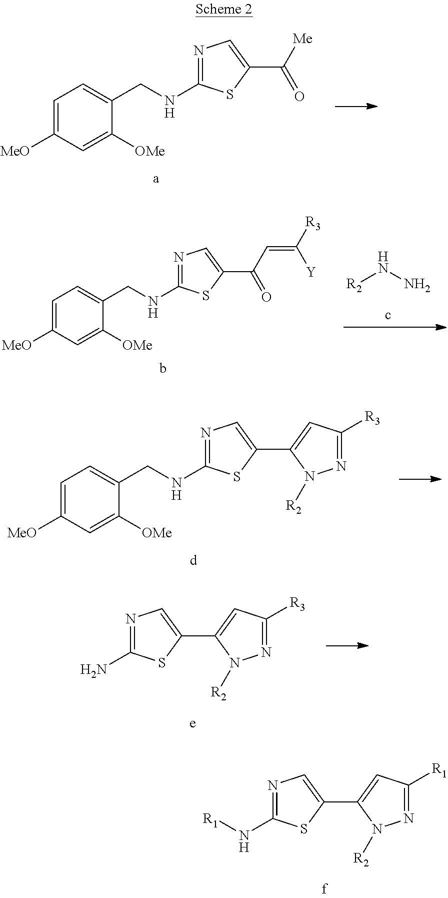

In the method represented in Scheme 2, N-(2,4-dimethoxy)-4-acetyl-2-aminothiazole (a) is carbonylated by heating in the presence of an appropriate electrophile (e.g., a substituted malonate or dimethylformamide dimethylacetal) and base (e.g., sodium ethoxide) to give compound (b). Condensation of enone (b) with substituted hydrazines of the formula R₂—NHNH₂ (c) provides pyrazole (d). Deprotection of compound (d) using wet acid (e.g., trifluoroacetic acid) at elevated temperatures provides 2-aminothiazole (e). 2-Aminothiazole (e) can then be transformed into compounds (f) via addition of an appropriate electrophile (e.g. acid chlorides, sulfonyl chlorides, isocyanates, or heteroaryl chlorides) or via conversion to the 2-bromo-aminothiazole (see Das, et al. J. Med. Chem. 2006, 49, 6819-6832) and displacement with a suitable nucleophiles (e.g. amines, alcohols, or anilines).

Hydrazines (c) of the formula R₂—NHNH₂ can be prepared from their corresponding amines according to methods known in the art. One approach is described in Finkelstein, et al. WO 2008124092, and is shown below in Scheme 3:

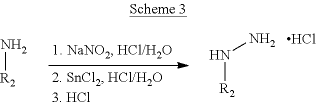

### 5.4. Methods of Use

This invention encompasses a method of inhibiting LIMK2, which comprises contacting LIMK2 with a potent LIMK2 inhibitor. Preferred potent LIMK2 inhibitors are compounds of the invention (i.e., compounds disclosed herein).

A particular embodiment encompasses a method of treating, managing or preventing an inflammatory disease or disorder in a patient, which comprises administering to the patient in need thereof a therapeutically or prophylactically effective amount of a compound of the invention.

Another embodiment encompasses a method of treating, managing or preventing cancer in a patient, which comprises administering to the patient in need thereof a therapeutically or prophylactically effective amount of a compound of the invention.

Another embodiment encompasses a method of lowering intraocular pressure in a patient, which comprises inhibiting LIMK2 activity or expression in a patient in need thereof. In one method, LIMK2 activity is inhibited by contacting the eye of the patient with a potent LIMK2 inhibitor. Particular potent LIMK2 inhibitors are disclosed herein. In another method, LIMK2 expression is inhibited by administering to the eye of the patient a compound (e.g., a siRNA) that inhibits the expression of LIMK2.

Another embodiment encompasses a method of treating, managing or preventing a disease or disorder affecting vision in a patient, which comprises inhibiting LIMK2 activity or expression in a patient in need thereof. In one method, LIMK2 activity is inhibited by contacting the eye of the patient with a potent LIMK2 inhibitor. Particular potent LIMK2 inhibitors are disclosed herein. Diseases and disorders affecting vision include glaucoma, neurodegenerative diseases, and infectious diseases.

### 5.5. Pharmaceutical Formulations

This invention encompasses pharmaceutical compositions comprising one or more compounds of the invention. Certain pharmaceutical compositions are single unit dosage forms suitable for oral, mucosal (e.g., nasal, sublingual, vaginal, buccal, or rectal), parenteral (e.g., subcutaneous, intravenous, bolus injection, intramuscular, or intraarterial), transdermal, topical and ophthalmic (e.g., topical, intravitreal) administration to a patient.

Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The formulation should suit the mode of administration. For example, oral administration requires enteric coatings to protect the compounds of this invention from degradation within the gastrointestinal tract. Similarly, a formulation may contain ingredients that facilitate delivery of the active ingredient(s) to the site of action. For example, compounds may be administered in liposomal formulations, in order to protect them from degradative enzymes, facilitate transport in circulatory system, and effect delivery across cell membranes to intracellular sites.

The composition, shape, and type of a dosage form will vary depending on its use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18^{th} ed. (Mack Publishing, Easton Pa.: 1990).

### 5.5.1. Oral Dosage Forms

Pharmaceutical compositions of the invention suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See, e.g., Remington's Pharmaceutical Sciences, 18^{th} ed. (Mack Publishing, Easton Pa.: 1990).

Typical oral dosage forms are prepared by combining the active ingredient(s) in an intimate admixture with at least one excipient according to conventional pharmaceutical compounding techniques. Excipients can take a wide variety of forms depending on the form of preparation desired for administration.

Because of their ease of administration, tablets and capsules represent the most advantageous oral dosage unit forms. If desired, tablets can be coated by standard aqueous or nonaqueous techniques. Such dosage forms can be prepared by conventional methods of pharmacy. In general, pharmaceutical compositions and dosage forms are prepared by uniformly and intimately admixing the active ingredients with liquid carriers, finely divided solid carriers, or both, and then shaping the product into the desired presentation if necessary. Disintegrants may be incorporated in solid dosage forms to facility rapid dissolution. Lubricants may also be incorporated to facilitate the manufacture of dosage forms (e.g., tablets).

### 5.5.2. Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are specifically sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 5.5.3. Transdermal, Topical and Mucosal Dosage Forms

Transdermal, topical, and mucosal dosage forms include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 18^{th} ed. (Mack Publishing, Easton Pa.: 1990); and Introduction to Pharmaceutical Dosage Forms, 4^{th} ed. (Lea & Febiger, Philadelphia: 1985). Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms are well known to those skilled in the pharmaceutical arts, and depend on the particular tissue to which a given pharmaceutical composition or dosage form will be applied.

Depending on the specific tissue to be treated, additional components may be used prior to, in conjunction with, or subsequent to treatment with active ingredients of the invention. For example, penetration enhancers may be used to assist in delivering active ingredients to the tissue.

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates may also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### 5.5.4. Ophthalmic Dosage Forms

Compounds of the invention can be delivered to the eye (e.g., topically) using aqueous solutions, aqueous suspensions, and ointments. As those skilled in the art are aware, the ophthalmic product must be sterile in its final container to prevent microbial contamination of the eye. Preservatives may be used to maintain sterility once the container has been opened. Ophthalmic formulations also require that the pH, buffer capacity, viscosity, and tonicity of the formulation be controlled. Preferred formulations have a pH of from about 6.5 to 8.5, and a buffer capacity of from about 0.01 to 0.1. Particular formations are isotonic. Particular formations have a viscosity of from about 25 to 50 cps.

Ingredients that may be used to provide safe vehicles that effectively deliver an active pharmaceutical ingredient (API) to its site of action are well known, but will vary depending on the physical and chemical characteristics of the API.

Appropriately buffered aqueous solutions may be used for the delivery of water soluble compounds. In solution compositions, polymeric ingredients are typically used to increase the composition's viscosity. Examples of suitable polymers include cellulosic polymers (e.g., hydroxypropyl methylcellulose, hydroxyethyl cellulose, ethylhydroxyethyl cellulose), synthetic polymers (e.g., carboxyvinyl polymers, polyvinyl alcohol), polysaccharides (e.g., xanthan gum, guar gum, and dextran), and mixtures thereof. See, e.g., U.S. Pat. Nos. 4,136,173 and 7,244,440. Suspensions may also be used to deliver compounds. Polymeric ingredients are typically used in suspension compositions as physical stability aids, helping to keep the insoluble ingredients suspended or easily redispersible. Id.

Preservatives may be used to ensure the sterility of formations. Suitable preservatives include benzalkonium chloride, benzethonium chloride, chlorobutanol, phenylmercuric acetate, phenylmercuric nitrate, thimerosal, methylparaben, and propyl-parabens. And antioxidants may be used to ensure the stability of formations susceptible to oxidation. Suitable antioxidants include ethylenediaminetetraacetic acid, sodium bisulfite, sodium metabisulfite, and thiourea.

### 6. EXAMPLES

Aspects of this invention can be understood from the following examples, which do not limit its scope.

### 6.1. Synthesis of N-(2,4-Dimethoxybenzyl)-4-acetyl-2-aminothiazole (3)

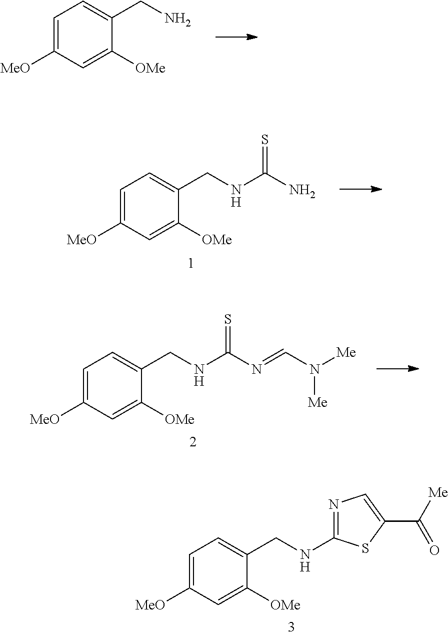 [SMILES: COc1ccc(CN)c(OC)c1 ; COc1ccc(CNC(N)=S)c(OC)c1 ; COc1ccc(CNC(=S)/N=C/N(C)C)c(OC)c1 ; COc1ccc(CNc2ncc(C(C)=O)s2)c(OC)c1]

1-(2,4-Dimethoxybenzyl)thiourea (1). 2,4-Dimethoxybenzylamine (15 mL, 99.8 mmol) was added over 30 minutes to a solution of 1,1′-thiocarbonyldiimidazole (90%, 21.8 g, 110 mmol) in 300 mL of dichloromethane via addition funnel. The reaction mixture was stirred for 3 hours at room temperature. A solution of methanolic ammonia (2N, 250 mL, 500 mmol) was added at the reaction was stirred for 72 hours. Volatiles were removed in vacuo. The resulting solids were slurried in 100 mL of dichloromethane and filtered. The precipitate was washed with excess dichloromethane to provide thiourea 1 as a tan solid (17.5 g, 77% yield). ¹H NMR (400 MHz, DMSO-d₆) δ: 7.66 (br. s., 1H), 7.12 (d, J=7.9 Hz, 1H), 7.00 (br. s., 2H), 6.55 (d, J=2.2 Hz, 1H), 6.49 (d, J=8.2 Hz, 1H), 4.46 (d, J=4.2 Hz, 2H), 3.79 (s, 3H), 3.74 (s, 3H). MS (ES+) [M+H]⁺: 227.2.

N′-(2,4-Dimethoxybenzylcarbamothioyl)-N,N-dimethylformimidamide (2). Dimethylformamide dimethylacetal (5.8 mL, 41.0 mmol) was added to a solution of 1-(2,4-dimethoxybenzyl)thiourea (1, 6.2 g, 27.3 mmol) in 30 mL of ethanol and heated for 1 hour at 80° C., at which temperature the reaction becomes a homogeneous solution and the reaction was deemed complete by LCMS analysis. A stream of nitrogen gas was passed over the reaction as it cooled to room temperature, causing a white solid to precipitate out. This solid was filtered and washed twice with 100 mL of ethanol to provide a 1:1 mixture of imine isomers as a white solid (6.55 g, 85% yield, 2.5:1 mixture of imidamide isomers). ¹H NMR (400 MHz, DMSO-d₆) δ: (major isomer): 8.79 (t, J=6.0 Hz, 1H), 8.70 (s, 1H), 7.00 (d, J=7.9 Hz, 1H), 6.53 (d, J=1.8 Hz, 1H), 6.43-6.46 (m, 1H), 4.61 (d, J=6.0 Hz, 2H), 3.78 (s, 3H), 3.73 (s, 3H), 3.13 (s, 3H), 2.99 (s, 3H). (minor isomer): 8.67 (t, J=6.0 Hz, 1H), 8.65 (s, 1H), 7.02 (d, J=7.7 Hz, 1H), 6.51 (d, J=2.2 Hz, 1H), 6.44-6.47 (m, 1H), 4.45 (d, J=6.2 Hz, 2H), 3.77 (s, 3H), 3.73 (s, 3H), 3.13 (s, 3H), 2.97 (s, 3H). MS (ES+) [M+H]⁺: 282.2.

N-(2,4-Dimethoxybenzyl)-4-acetyl-2-aminothiazole (3). Chloroacetone (1.21 mL, 15.2 mmol) was added to formimidamide 2 (3.56 g, 12.7 mmol) in 32 mL of acetonitrile. This mixture was heated to 75° C. for 3 hours. A stream of nitrogen gas was passed over the reaction as it cooled to room temperature until the initial reaction volume was decreased by half. 37.5 mL of water and 12.5 mL of saturated aqueous NaHCO₃ was added and the slurry was stirred for 15 minutes. The precipitate was filtered and was with 100 mL each of water and 20% diethyl ether/hexanes providing acetylthiazole 3 as an off-white solid (3.49 g, 95% yield) after drying under vacuum. ¹H NMR (DMSO-d₆): 8.85 (br. s., 1H), 7.97 (s, 1H), 7.15 (d, J=8.4 Hz, 1H), 6.57 (d, J=2.2 Hz, 1H), 6.48 (dd, J=8.4, 2.2 Hz, 1H), 4.37 (d, J=5.1 Hz, 2H), 3.79 (s, 3H), 3.74 (s, 3H), 2.34 (s, 3H). MS (ES+) [M+H]⁺: 371.1, [M+H+H₂O]⁺: 389.1.

### 6.2. Synthesis of 5-(1-(2,6-Dichlorophenyl)-3-(difluoromethyl)-1H-pyrazol-5-yl)thiazol-2-amine (6)

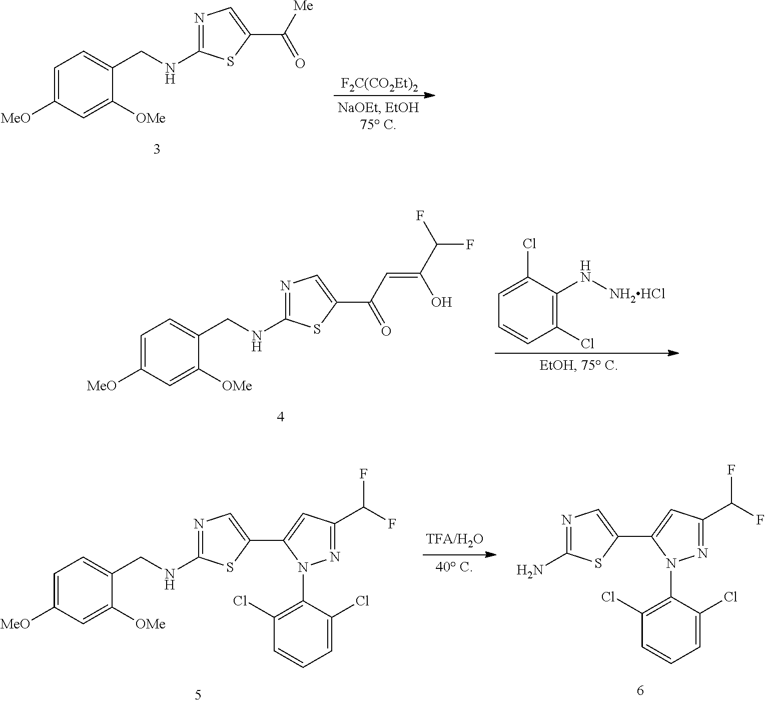

1-(2-(2,4-Dimethoxybenzylamino)thiazol-5-yl)-4,4-difluorobutane-1,3-dione (4). A mixture of aminothiazole 3 (1.11 g, 3.80 mmol) and diethyl 2,2-difluoromalonate (2.98 g, 15.2 mmol) in a solution of sodium ethoxide in ethanol (approx. 3 N, 5.4 mL) was heated to 75° C. for 5 hours, after which the reaction had turned homogeneous. After cooling to room temperature, the reaction mixture was transferred to a flask with excess water. The pH was adjusted to between 5 and 6 using glacial acetic acid. The resulting solid was filtered and washed with 100 mL each of water/methanol (2:1 v:v) and diethyl ether/hexanes (1:4 v:v). The solid was dried under vacuum overnight to provide the title compound as an orange solid (1.05 g, 74% yield). ¹H NMR (DMSO-d₆) δ: 9.26 (br. s., 1H), 8.33 (s, 1H), 7.16 (d, J=8.3 Hz, 1H), 6.57-6.61 (m, 2H), 6.50 (dd, J=8.3, 2.5 Hz, 1H), 4.41 (br. s., 2H), 3.80 (s, 3H), 3.75 (s, 3H), 3.35 (br. s., 1H). ¹⁹F NMR (DMSO-d₆) δ: −126.70 (d, J=53.9 Hz). MS (ES+) [M+H]⁺: 293.1.

5-(1-(2,6-Dichlorophenyl)-3-(difluoromethyl)-1H-pyrazol-5-yl)-N-(2,4-dimethoxy-benzyl)thiazol-2-amine (5). A mixture of ketone 4 (296 mg, 0.800 mmol) and 2,6-dichlorophenylhydrazine hydrochloride (205 mg, 0.960 mmol) in 6 mL of ethanol was heated to 75° C. for 2 hours. The reaction was cooled and treated with 4 mL of water and 1.5 mL of saturated aqueous NaHCO₃, resulting in the formation of a precipitate. The precipitate was filter and washed with 10 mL each of water/methanol (2:1 v:v) and diethyl ether/hexanes (1:4 v:v). The solid was dried under vacuum overnight to provide pyrazole 5 as an beige solid (358 mg, 88% yield). ¹H NMR (DMSO-d₆) 6: 8.12 (t, J=5.7 Hz, 1H), 7.77 (d, J=8.8 Hz, 1H), 7.77 (d, J=7.1 Hz, 1H), 7.70 (dd, J=9.4, 6.6 Hz, 1H), 7.21 (s, 1H), 7.07 (d, J=3.8 Hz, 1H), 7.01 (t, J=32.1 Hz, 1H), 6.53 (d, J=2.5 Hz, 1H), 6.45 (dd, J=8.3, 2.5 Hz, 1H), 4.25 (d, J=5.6 Hz, 2H), 3.76 (s, 3H), 3.73 (s, 3H). ¹⁹F NMR (DMSO-d₆) δ: −112.52 (d, J=53.9 Hz). MS (ES+) [M+H]⁺: 511.0.

5-(1-(2,6-Dichlorophenyl)-3-(difluoromethyl)-1H-pyrazol-5-yl)thiazol-2-amine (6). To a vial charged with pyrazole 5 (358 mg, 0.703 mmol) was added 0.5 mL of water and 5 mL of trifluoroacetic acid. The reaction turns a bright pink color as it progresses. After 4 hours, the reaction was diluted with 20 mL of water and neutralized with saturated aqueous NaHCO₃. The solids were filtered, washed with water, and further purified by silica gel chromatography (gradient 50% to 100% ethyl acetate/hexanes) to provide 2-aminothiazole 6 as an light orange amorphous solid (237 mg, 94% yield). ¹H NMR (METHANOL-d₄) δ: 7.57-7.68 (m, 3H), 6.93 (s, 1H), 6.86 (s, 1H), 6.80 (t, J=54.6 Hz, 1H).). ¹⁹F NMR (METHANOL-d₄) δ: −114.37 (d, J=55.1 Hz). MS (ES+) [M+H]⁺: 361.1.

### 6.3. Synthesis of N-(5-(1-(2,6-dichlorophenyl)-3-(difluoromethyl)-1H-pyrazol-5-yl)thiazol-2-yl)butyramide

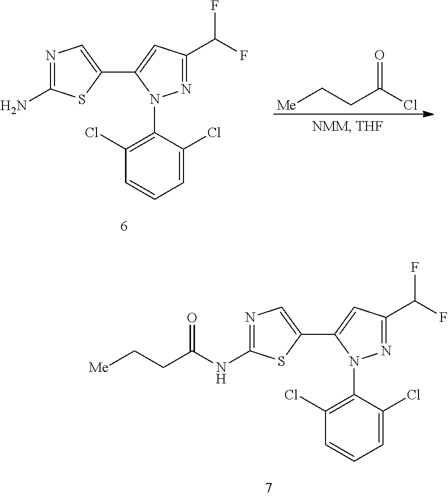 [SMILES: Nc1ncc(-c2cc(C(F)F)nn2-c2c(Cl)cccc2Cl)s1 ; CCCC(=O)Cl ; CCCC(=O)Nc1ncc(-c2cc(C(F)F)nn2-c2c(Cl)cccc2Cl)s1]

N-(5-(1-(2,6-Dichlorophenyl)-3-(difluoromethyl)-1H-pyrazol-5-yl)thiazol-2-yl)butyramide (7). To a solution of aminothiazole 6 (20 mg, 0.055 mmol) in THF (0.5 mL) was added N-methylmorpholine (0.030 mL, 0.278 mmol) followed by butyryl chloride (0.030 mL, 0.278 mmol). The reaction was stirred for 5 minutes. The reaction was filtered, concentrated in vacuo, and purified by preparative HPLC ((30×100mm C18 column, 10-100% methanol:water (10 mM ammonium acetate), 15 min, 45 mL/min) to afford the desired amide 7 (9.5 mg, 40% yield, 98.9% pure by HPLC analysis). ¹H NMR (METHANOL-d₄) δ: 7.57-7.69 (m, 3H), 7.37 (s, 1H), 7.00 (s, 1H), 6.83 (t, J=54.8 Hz, 1H), 2.41 (t, J=7.5 Hz, 2H), 1.63-1.75 (m, 2H), 0.96 (t, J=7.5 Hz, 3H). ¹⁹F NMR (METHANOL-d₄) δ: −114.40 (d, J=55.1 Hz). MS (ES+) [M+H]⁺: 431.0.

### 6.4. Synthesis of N-(5-(1-(2,6-dichlorophenyl)-3-(difluoromethyl)-1H-pyrazol-5-yl)thiazol-2-yl)-4-(dimethylamino)butanamide

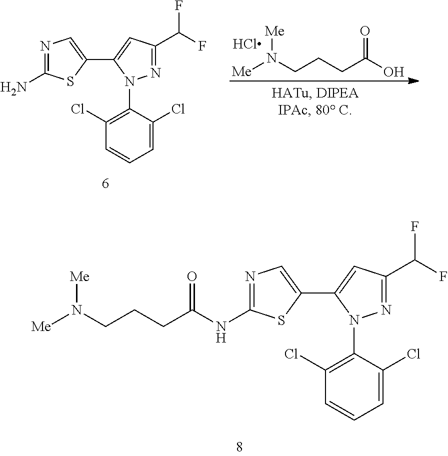 [SMILES: Nc1ncc(-c2cc(C(F)F)nn2-c2c(Cl)cccc2Cl)s1 ; CN(C)CCCC(=O)O ; CN(C)CCCC(=O)Nc1ncc(-c2cc(C(F)F)nn2-c2c(Cl)cccc2Cl)s1]

N-(5-(1-(2,6-Dichlorophenyl)-3-(difluoromethyl)-1H-pyrazol-5-yl)thiazol-2-yl)-4-(dimethylamino)butanamide (8). To a solution of aminothiazole 6 (80 mg, 0.22 mmol) in iso-propyl acetate (2.0 mL) was added N,N-diisopropylethylamine (0.19 mL, 1.1 mmol), 4-(dimethylamino)butanoic acid hydrochloride (93 mg, 0.55 mmol), and HATu (211 mg, 0.55 mmol). The reaction was heated at 80° C. for 1 h, after which it was quenched with a 1:1 (v:v) mixture of saturated aqueous NH₄Cl/brine. The aqueous layer was extracted with ethyl acetate. The combined organic layers were washed with brine, dried with magnesium sulfate, filtered and concentrated. The crude residue was purified by preparative HPLC ((30×100 mm C18 column, 10-100% methanol:water (10 mM ammonium formate), 15 min, 45 mL/min) to afford the desired amide (8, 65 mg, 62% yield). ¹H NMR (METHANOL-d₄) δ: 7.57-7.69 (m, 3H), 7.39 (s, 1H), 7.01 (s, 1H), 6.84 (t, J=54.8 Hz, 1H), 3.06-3.15 (m, 2H), 2.84 (s, 6H), 2.59 (t, J=6.9 Hz, 2H), 1.99-2.10 (m, 2H). ¹⁹F NMR (METHANOL-d₄) δ: −114.37 (d, J=54.9 Hz). MS (ES+) [M+H]⁺: 474.0.

### 6.5. Synthesis of (S)—N-(5-(1-(2,6-dichlorophenyl)-3-(difluoromethyl)-1H-pyrazol-5-yl)thiazol-2-yl)-2-(pyrrolidin-2-yl)acetamide

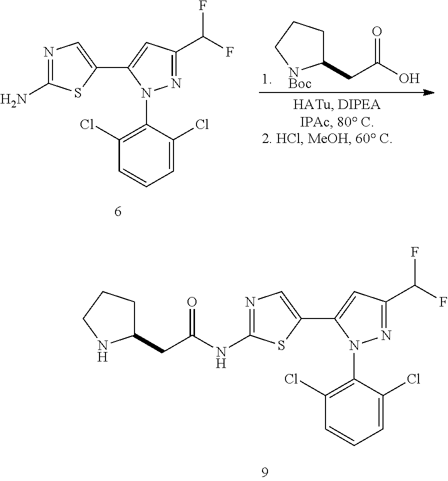

(S)—N-(5-(1-(2,6-dichlorophenyl)-3-(difluoromethyl)-1H-pyrazol-5-yl)thiazol-2-yl)-2-(pyrrolidin-2-yl)acetamide (9). To a solution of aminothiazole 6 (25 mg, 0.069 mmol) in iso-propyl acetate (0.5 mL) was added N,N-diisopropylethylamine (0.060 mL, 0.35 mmol), (S)-2-(1-tert-butoxycarbonyl)pyrrolidin-2-yl)acetic acid hydrochloride (39 mg, 0.17 mmol), and HATu (65 mg, 0.17 mmol). The reaction was heated at 80° C. for 1 h, after which it was quenched with a 1:1 (v:v) mixture of saturated aqueous NH₄Cl/brine. The aqueous layer was extracted with ethyl acetate. The combined organic layers were washed with brine, dried with magnesium sulfate, filtered and concentrated. The crude residue was taken up in 1 mL of methanol and 0.2 mL of 4N HCl in dioxane was added. The reaction was heated at 60° C. for 1 h. Volatiles were removed in vacuo and the crude residue was purified by preparative HPLC ((30×100 mm C18 column, 10-100% methanol:water (10 mM ammonium formate), 15 min, 45 mL/min) to afford the desired amide as the mono-formate salt (9, 13.0 mg, 36% yield). ¹H NMR (methanol -d₄) δ: 8.47 (s, 1H), 7.56-7.69 (m, 3H), 7.45 (s, 1H), 7.02 (s, 1H), 6.84 (t, J=54.6 Hz, 1H), 3.85-3.96 (m, 1H), 3.06 (dd, J=17.6, 3.7 Hz, 1H), 2.81-2.93 (m, 1H), 2.22-2.34 (m, 1H), 2.04-2.15 (m, 1H), 1.92-2.04 (m, 1H), 1.65-1.79 (m, 1H). ¹³C NMR (DMSO-d₆) δ: 168.7, 158.7, 148.2 (t, J=29.3 Hz), 138.4, 138.3, 134.4, 133.8, 133.5, 129.5, 116.6, 111.0 (t, J=232.7 Hz), 103.2, 54.8, 44.6, 36.8, 29.8, 23.0. ¹⁹F NMR (methanol-d₄) δ: −114.38 (d, J=55.1 Hz). MS (ES+) [M+H]⁺: 472.1.

### 6.6. Synthesis of 5-(1-(2,6-dichlorophenyl)-1H-pyrazol-5-yl)thiazol-2-amine (13)

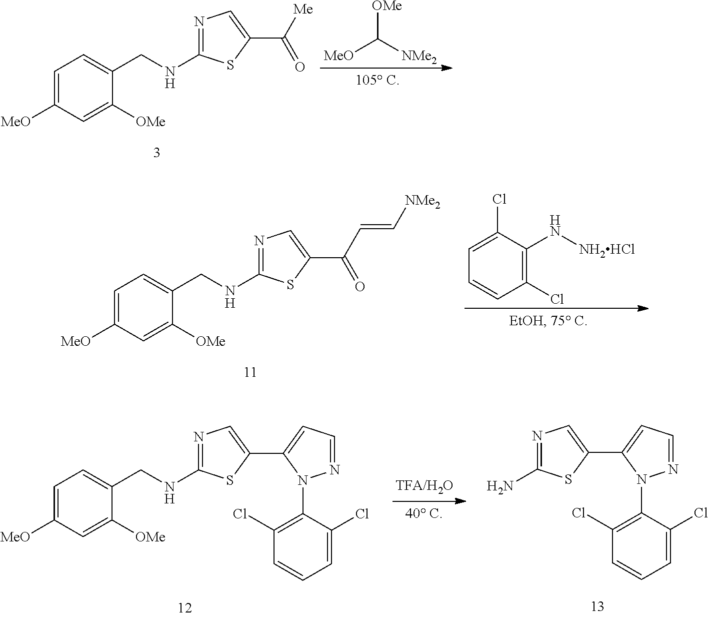

1-(2-(2,4-Dimethoxybenzylamino)thiazol-5-yl)-3-(dimethylamino)prop-2-en-1-one (11). Ketone 3 (2.25 g, 7.70 mmol) in 22.5 mL of dimethylformamide dimethylacetal was heated to 105° C. for 18 hours. The solution was cooled to ambient temperature. 2.5 mL of ethanol was added, followed by 67 mL of diethyl ether. This precipitated mixture was stirred for 30 minutes and filtered. The precipitate was washed with excess diethyl ether and dried under vacuum for 18 hours to provide enone 11 as an off-white solid (1.00 g, 37% yield). ¹H NMR (DMSO-d₆) δ: 8.38 (t, J=5.7 Hz, 1H), 7.77 (s, 1H), 7.47 (d, J=12.3 Hz, 1H), 7.15 (d, J=8.2 Hz, 1H), 6.56 (d, J=2.2 Hz, 1H), 6.48 (dd, J=8.3, 2.3 Hz, 1H), 5.58 (d, J=12.6 Hz, 1H), 4.32 (d, J=5.5 Hz, 2H), 3.80 (s, 3H), 3.74 (s, 3H), 3.33 (s, 6H). MS (ES−) [M−H]: 346.1.

5-(1-(2,6-Dichlorophenyl)-1H-pyrazol-5-yl)-N-(2,4-dimethoxybenzyl)thiazol-2-amine (12). A mixture of ketone 11 (347 mg, 1.00 mmol) and 2,6-dichlorophenylhydrazine hydrochloride (256 mg, 1.20 mmol) in 7 mL of ethanol was heated to 75° C. for 2 hours. The reaction was cooled and treated with 5 mL of water and 1.5 mL of saturated aqueous NaHCO₃, resulting in the formation of a precipitate. The precipitate was filter and washed with 10 mL each of water/methanol (2:1 v:v) and diethyl ether/hexanes (1:4 v:v). The solid was dried under vacuum and used without further purification in the next step. MS (ES+) [M+H]⁺: 461.1.

5-(1-(2,6-Dichlorophenyl)-1H-pyrazol-5-yl)thiazol-2-amine (13). To a vial charged with pyrazole 12 (460 mg, 1.00 mmol) was added 1.5 mL of water and 7 mL of trifluoroacetic acid. The reaction turns a bright pink color as it progresses. After 4 hours, the reaction was diluted with 20 mL of water and neutralized with saturated aqueous NaHCO₃. The solids were filtered, washed with water, and further purified by silica gel chromatography (gradient 40% to 80% ethyl acetate/hexanes) to provide 2-aminothiazole 13 as an light yellow solid (216 mg, 70% yield for 2 steps). ¹H NMR (methanol-d₄) δ: 7.76 (d, J=2.0 Hz, 1H), 7.55-7.66 (m, 3H), 6.84 (s, 1H), 6.64 (d, J=2.0 Hz, 1H). MS (ES+) [M+H]⁺: 311.1.

### 6.7. Synthesis of N-(5-(1-(2.6-dichlorophenyl)-1H-pyrazol-5-yl)thiazol-2-yl)butyramide (14)

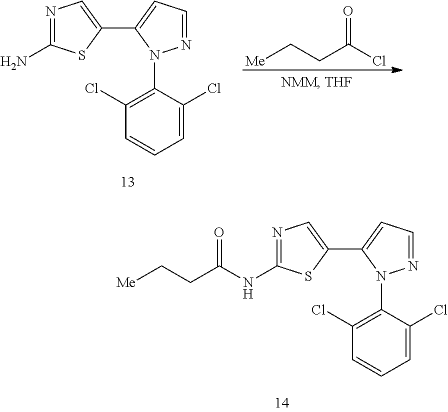 [SMILES: Nc1ncc(-c2ccnn2-c2c(Cl)cccc2Cl)s1 ; CCCC(=O)Cl ; CCCC(=O)Nc1ncc(-c2ccnn2-c2c(Cl)cccc2Cl)s1]

N-(5-(1-(2,6-dichlorophenyl)-1H-pyrazol-5-yl)thiazol-2-yl)butyramide (14). To a solution of aminothiazole 13 (35 mg, 0.11 mmol) in THF (1.0 mL) was added N-methylmorpholine (0.12 mL, 1.1 mmol) followed by butyryl chloride (0.12 mL, 1.1 mmol). The reaction was stirred for 5 minutes. The reaction was filtered, concentrated in vacuo, and purified by preparative HPLC ((30×100mm C18 column, 10-100% methanol:water (10 mM ammonium acetate), 15 min, 45 mL/min) to afford the desired amide 14 (7.4 mg, 18% yield, 97.6% pure by HPLC analysis). ¹H NMR (METHANOL-d₄) δ: 7.83 (d, J=2.0 Hz, 1H), 7.55-7.68 (m, 3H), 7.28 (s, 1H), 6.79 (d, J=2.0 Hz, 1H), 2.41 (t, J=7.4 Hz, 2H), 1.62-1.76 (m, 2H), 0.96 (t, J=7.4 Hz, 3H) MS (ES+) [M +H]⁺: 381.1.

### 6.8. Synthesis of 4-acetamido-N-(5-(1-(2,6-dichlorophenyl)-3-(difluoromethyl)-1H-pyrazol-5-yl)thiazol-2-yl)butanamide (15).

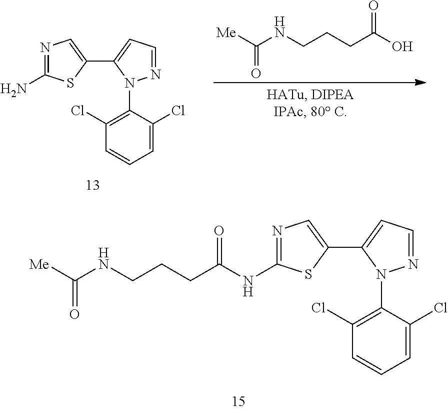 [SMILES: Nc1ncc(-c2ccnn2-c2c(Cl)cccc2Cl)s1 ; CC(=O)NCCCC(=O)O ; CC(=O)NCCCC(=O)Nc1ncc(-c2ccnn2-c2c(Cl)cccc2Cl)s1]

4-Acetamido-N-(5-(1-(2,6-dichlorophenyl)-3-(difluoromethyl)-1H-pyrazol-5-yl)thiazol-2-yl)butanamide (15). To a solution of aminothiazole 13 (20 mg, 0.064 mmol) in iso-propyl acetate (0.5 mL) was added N,N-diisopropylethylamine (0.057 mL, 0.32 mmol), 4-acetamidobutanoic acid (28 mg, 0.19 mmol), and HATu (94 mg, 0.21 mmol). The reaction was heated at 80° C. for 1 h, after which it was quenched with a 1:1 (v:v) mixture of saturated aqueous NH₄Cl/brine. The aqueous layer was extracted with ethyl acetate. The combined organic layers were washed with brine, dried with magnesium sulfate, filtered and concentrated. The crude residue was purified by preparative HPLC ((30×100mm C18 column, 10-100% methanol:water (10 mM ammonium acetate), 15 min, 45 mL/min) to afford the desired amide (15, 8.5 mg, 30% yield, 95% pure by HPLC analysis). ¹H NMR (METHANOL-d₄) δ: 7.82 (d, J=2.0 Hz, 1H), 7.55-7.68 (m, 3H), 7.27 (s, 1H), 6.78 (d, J=2.0 Hz, 1H), 3.21 (t, J=6.8 Hz, 2H), 2.47 (t, J=7.3 Hz, 2H), 1.90 (s, 3H), 1.85 (t, J=7.1 Hz, 2H). MS (ES+) [M+H]⁺: 438.0.

### 6.9. Synthesis of (2,6-dimethylphenyl)hydrazine hydrochloride

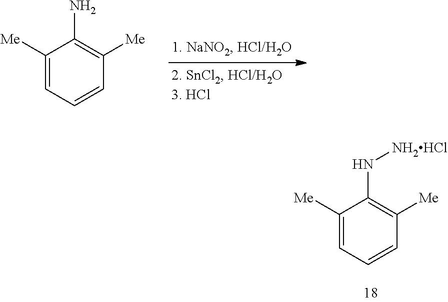

(2,6-Dimethylphenyl)hydrazine hydrochloride (18). A 100-mL round-bottomed flask equipped with an addition funnel was charged with 6.25 mL of concentrated HCl and 5 mL of water. The solution was cooled to −5° C. 2,6-Dimethylaniline (3.5 mL, 28.2 mmol) was added dropwise via syringe, forming a white precipitate. This mixture was stirred for another 15 minutes. A solution of sodium nitrite (1.95 g, 28.2 mmol) in 5 mL of water was added dropwise via syringe at −5° C., causing the white mixture to turn orange. After 30 minutes, a solution of tin(II) chloride (13.4 g, 70.5 mmol) in 23 mL of a 1:1 (v:v) solution of concentrated HCl/water was added dropwise over 1 hour via addition funnel. The reaction was stirred vigorously at ambient temperature overnight. The resulting precipitate was filtered and washed sequentially with brine and diethyl ether. The preciptate was then added to a flask charged with 35 mL of diethyl ether and 50 mL 10N aqueous NaOH at 0° C. The mixture was stirred at ambient temperature until the solids dissolved. The layers were separated and the aqueous layer was extracted two times with 50 mL of diethyl ether. The combined ether layers were cooled to 0° C. and 4N HCl in dioxane (6.25 mL) was added dropwise and the reaction was stirred for 30 minutes. The resulting white solid was filtered, washed with cold diethyl ether and dried under vacuum to provide the mono-HCl salt of hydrazine 18 (1.24 g, 25% yield).

### 6.10. Synthesis of (2,6-dimethyl-4-cyanophenyl)hydrazine hydrochloride

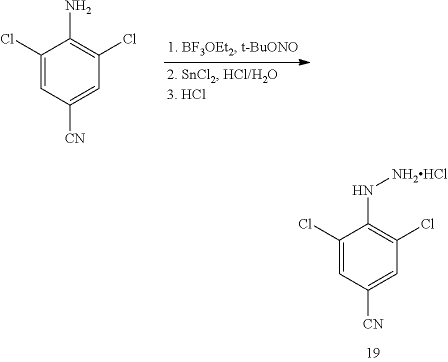

(2,6-Dichloro-4-cyanophenyl)hydrazine hydrochloride (19). A flask charged with a solution of 2,6-dichloro-4-cyanoaniline (5.00 g, 26.7 mmol) in 81 mL of THF was cooled to 0° C. Boron trifluoride diethyletherate (5.03 mL, 40.1 mmol) was added dropwise via syringe, followed by the dropwise addition of tert-butyl nitrite (3.80 mL, 32.0 mmol). This reaction was stirred for another 60 minutes, over which time a tan precipitate formed. 100 mL of diethyl ether was added and the mixture was stirred for 30 minutes. The precipitate was filtered and washed with excess diethyl ether. The diazonium salt was isolated as a tan solid (5.57 g, 78% yield) and used in the subsequent reduction without further purification. ¹H NMR (DMSO-d₆) δ: 8.31 (s, 2H). Product does not ionize in the mass spectrometer.

The diazonium salt (5.57 g, 20.8 mmol) from the previous step was suspended in 52 mL of a 1:1 (v:v) solution of concentrated HCl/water and cooled to 0° C. Tin(II) chloride (9.85 g, 52.0 mmol) was added in 500 mg portions. The reaction was stirred at room temperature for 45 hours. The resulting precipitate was filtered and washed sequentially with brine and diethyl ether. The preciptate was then added to a flask charged with 100 mL of diethyl ether and 100 mL 6N aqueous NaOH. The mixture was stirred at ambient temperature for 3 hours. The layers were separated and the aqueous layer was extracted 100 mL of diethyl ether and 50 mL of ethyl acetate. The combined organic layers were concentrated. The crude residue was taken up in 150 mL of diethyl ether and 20 mL of ethyl acetate at 0° C. 4N HCl in dioxane (8.0 mL) was added dropwise and the reaction was stirred for 30 minutes at 0° C. and allowed to settle at 0° C. overnight. The tan solid was collected by filtration, washed with cold diethyl ether and dried under vacuum to provide hydrazine 19 as mostly the mono-HCl salt (928 mg, 19% yield, about 70% pure by 1H NMR analysis). ¹H NMR (DMSO-d₆) δ: 8.09 (s, 2H). MS (ES−) [M−H]: 200.1.

### 6.11. Expression and Purification of LIMK2

LIMK2 was expressed using the BAC-to-BAC® Baculovirus Expression System (Invitrogen). Recombinant baculovirus was made according to the manufacturer's directions as set forth in the instruction manual. Briefly, the plasmids (pFactBac1 or pFastBacHT) carrying the LIMK2 inserts were transformed into MAX efficiency DH10Bac competent E. coli to generate a recombinant bacmid. The DH10Bac E. coli host strain contains a baculovirus shuttle vector (bacmid) with a mini-attTn7 target site and a helper plasmid, and allows generation of a recombinant bacmid following transposition between the mini-Tn7 element on the pFastBac vector and the min-attTn7 target site on the bacmid. The transposition reaction occurs in the presence of transposition proteins supplied by the helper plasmid. Cells were plated and the white colonies picked for bacmid isolation as described in the instruction manual.

The isolated bacmid DNA was transfected into SF9 cells to generate a recombinant baculovirus, and virus was collected five days after transfection. Virus was amplified in T75 flasks at a multiplicity of infection (MOI) of 0.2. The amplified virus was used to infect SF9 cells at a MOI 5 for protein expression.

For small scale purification of the LIMK2 constructs, a 50 ml culture of Sf9 cells infected with the recombinant baculovirus was used. The cells were harvested by centrifugation for 5 minutes at 500×g. The cells were then resuspended in lysis buffer (5 volumes per gram of cells). A typical lysis buffer contains the following: 50 mM HEPES (pH 8.0), 300 mM KCl, 10% glycerol, 1% NP-40, 15 mM imidazole, 1 mM benzamidine, and Roche complete protease inhibitors (1 tablet per 50 ml of cell lysate). The cellular suspension was lysed by one passage through a Microfluidics Microfluidizer M-110Y at a liquid pressure of 14,000 to 20,000 psi followed by centrifugation of the lysate at 60,000×g for 15 minutes at 4° C.

The supernatant was then loaded directly onto a chromatography matrix containing Cobalt ion covalently attached to nitrilotriacetic acid NTA. The chromatography matrix was equilibrated in the same buffer as the protein loading solution. The ion charged resin typically has a binding capacity equivalent to 5 to 10 mg histidine-tagged protein per ml of packed resin. The amount of extract that can be loaded onto the column depends on the amount of soluble histidine-tagged protein in the extract. The column was then washed in a stepwise fashion, first with: 50 mM HEPES (pH 8.0), 300 mM KCl, 10% glycerol, 1% NP-40, 15 mM imidazole, 1 mM benzamidine; second, with 20 mM HEPES (pH 8.0), 500 mM KCl, 10% glycerol, and 20 mM imidazole; third, with 20 mM HEPES (pH 8.0), 100 mM KCl, 10% glycerol, and 20 mM imidazole; followed by elution with 250 mM imidazole in the same buffer. The LIMK2 protein solution was then analyzed by SDS-PAGE and Western blot using commercial antibodies directed to both the carboxyl terminus and internal catalytic domains of the protein. For storage purposes the protein was dialyzed into 50 mM Tris (pH 7.5), 150 mM NaCl, 0.1% BME, 0.03% Brij-35, and 50% glycerol.

Large scale LIMK2 purification was done in a Wave Bioreactor (Wave Biotech) with 10 L culture volumes. 10 L of cell culture at 2-3×10⁶ viable cells/mL were infected at an MOI=5 pfu/cell and harvested at 48 hours post infection.

### 6.12. In Vitro LIMK2 Inhibition Assay

An in vitro assay used to identify LIMK2 inhibitors was developed. The analytical readout was the incorporation of ³³P from ATP substrate into immobilized myelin basic protein coated flash plates (Perkin Elmer Biosciences), which were counted on a scintillation counter equipped with a plate reader (TopCount, Packard Bioscience, Meriden, Conn.). Using 384 well flat MBP flashplates, total assay volume was 50 μl. The HTS program utilized a Biomek FX for dilution.

For each assay, the ingredients and conditions were as follows: 200 ng of enzyme was incubated in assay buffer (1× assay buffer contains 30 mM HEPES (pH 8.0), 5 mM DTT, and 10 mM MgCl₂), 10 μM ATP, 0.2 μCi [gamma-³³P]-ATP and 10 μM of potential inhibitory compound. The reaction was incubated at room temperature for 60 minutes, washed 3 times with 75 μl of stop/wash buffer (1× stop/was buffer contains 50 mM EDTA and 20 mM Tris (pH 7.4)), and then the plates were read on the scintillation counter. Different concentrations of staurosporine (400 nM, 200 nM, 100 nM and 50 nM; purchased from BIOMOL (Plymouth Meeting, Pa.)) were used as controls on each plate.

### 6.13. Dexamethasone-Induced Ocular Hypertension Model

Twenty eight day mouse Alzet mini-osmotic pumps (DURECT Corp., Cupertino, Calif.) were filled with a solution of water soluble dexamethasone (dex) in PBS (Sigma, St. Louis, Mo.) so that they would release roughly 0.1 mg of dex per day. Once the pumps were filled with the dex, the pumps were allowed to equilibrate in PBS at 37° C. for 60 hours. The equilibrated pumps were surgically placed subcutaneously on the backs of wild-type C57:129 F2 hybrid mice weighing between 25 and 35 grams. Surgical incisions were sutured with 5-0 braided silk (ROBOZ, Gaithersburg, Md.) and treated with antibiotic ointment throughout the entire duration of study. Surgical incisions were glued with TissueMend II (Webster Veterinary, Houston, Tex.). Analgesic (buprenorphine) was given through IP injection the day of surgery and 24 hours after surgery. Intraocular pressure (IOP) was measured on these mice using a TonoLab (Colonial Medical Supply Co., Franconia, N.H.) tonometer. Mice were mildly sedated with isoflurane and topically anesthetized with 0.5% proparacaine (Akorn, Buffalo Grove, Ill.) before IOP measurements were taken. Baseline IOP was measured 1 day prior to mini-pump implantation. After mini-pump implantation, IOP measurements were taken 2-3 times per week for 4 weeks. Pharmacology studies with potential ocular hypotensive compounds were performed between 21 and 28 days after implantation.

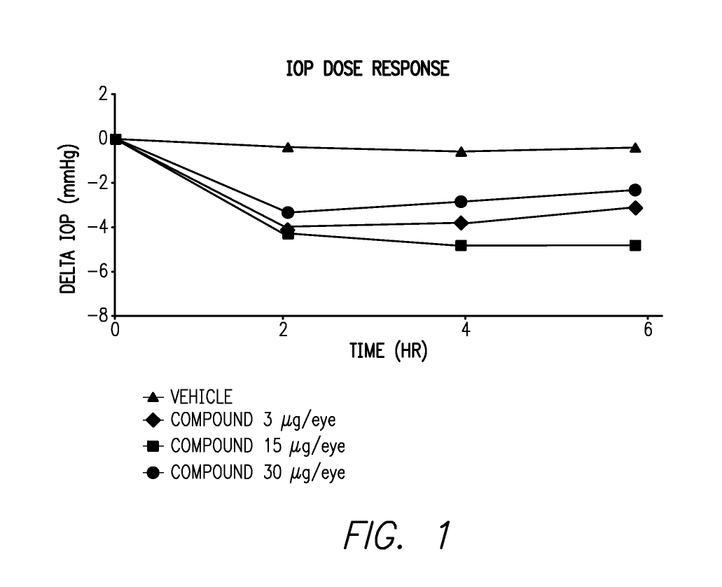

FIG. 1 shows the dose dependent effect of (S)—N-(5-(1-(2,6-dichlorophenyl)-3-(difluoromethyl)-1H-pyrazol-5-yl)thiazol-2-yl)-2-(pyrrolidin-2-yl)acetamide in this model. Average changes in IOP measured from time of dosing are provided in Table 1.

TABLE 1

| | 0 hr | 2 hr | 4 hr | 6 hr |
|---|---|---|---|---|
| Vehicle | 0.00 (0.0) | −0.33 (0.5) | −0.52 (0.6) | −0.33 (0.7) |
| Compound | 0.00 (0.0) | −4.10 (2.6) | −3.81 (3.8) | −3.19 (3.2) |
| 3 μg/eye | | | | |
| Compound | 0.00 (0.0) | −4.25 (2.7) | −4.88 (5.1) | −4.88 (4.2) |
| 15 μg/eye | | | | |
| Compound | 0.00 (0.0) | −3.33 (4.2) | −2.86 (1.1) | −2.29 (2.2) |
| 30 μg/eye | | | | |

All publications (e.g., patents and patent applications) cited above are incorporated herein by reference in their entireties.

What is claimed is:

1. A formulation suitable for ophthalmic administration, which comprises a liquid vehicle and a compound of the formula: 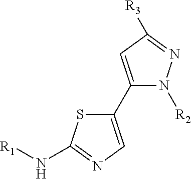
or a pharmaceutically acceptable salt thereof, wherein:
R₁ is H, C(O)R_{A}, S(O)ₙR_{A}, C(O)NR_{A}R_{B}, S(O)ₙNR_{A}R_{B}, S(O)ₙOR_{A}, C(NH)NR_{A}R_{B}, C(O)OR_{A}, C(S)NR_{A}R_{B}, C(SR_{B})NR_{A}, P(O)(OR_{A})₂or optionally substituted alkyl, aryl, or heterocycle (e.g., optionally substituted with halo, alkyl, alkoxyl, aryl, heteroaryl, hydroxyl, cyano, NR_{A}R_{B}, SR_{A}, P(O)(OR_{A})₂, CO₂R_{A}, C(O)NR_{A}R_{B}, S(O)ₙR_{A}, S(O)NR_{A}R_{B}, or halogenated (e.g., fluorinated) alkyl, aryl or heteroaryl);
R₂ is H, C(O)R_{A}, S(O)ₙR_{A}, C(O)NR_{A}R_{B}, S(O)ₙNR_{A}R_{B}, S(O)ₙOR_{A}, or optionally substituted alkyl, aryl, or heterocycle (e.g., optionally substituted with halo, alkyl, alkoxyl, aryl, heteroaryl, hydroxyl, cyano, NR_{A}R_{B}, SR_{A}, P(O)(OR_{A})₂, CO₂R_{A}, C(O)NR_{A}R_{B}, S(O)ₙR_{A}, S(O)NR_{A}R_{B}, or halogenated (e.g., fluorinated) alkyl, aryl or heteroaryl);
R₃ is H, halogen, OR, NR_{A}R_{B}, optionally substituted alkyl (e.g., optionally substituted with halo, alkyl, alkoxyl, hydroxyl, cyano, NR_{A}R_{B}, SR_{A}, CO₂R_{A}, C(O)NR_{A}R_{B};
each R_{A} is independently H or optionally substituted alkyl, aryl, alkylaryl, or alkyl-heterocycle (e.g., optionally substituted with halo, alkyl, alkoxyl, aryl, heteroaryl, hydroxyl, cyano, NR_{A}R_{B}, SR_{A}, P(O)(OR_{A})₂, CO₂R_{A}, C(O)NR_{A}R_{B}, S(O)ₙR_{A}, S(O)NR_{A}R_{B}, or halogenated (e.g., fluorinated) alkyl, aryl or heteroaryl);
each R_{B} is optionally substituted alkyl or aryl (e.g., optionally substituted with halo, alkyl, alkoxyl, aryl, heteroaryl, hydroxyl, cyano, NR_{A}R_{B}, SR_{A}, P(O)(OR_{A})₂, CO₂R_{A}, C(O)NR_{A}R_{B}, S(O)ₙR_{A}, S(O)NR_{A}R_{B}, or halogenated (e.g., fluorinated) alkyl, aryl or heteroaryl);
or when R_{A} and R_{B} are attached to the same nitrogen atom, they can be taken together with that nitrogen atom to form an optionally substituted heterocycle (e.g., piperidinyl, morpholino, thiomorpholino, piperazinyl, pyrrolidino, and azetidino optionally substituted with halo, alkyl, alkoxyl, aryl, heteroaryl, hydroxyl, cyano, NR_{A}R_{B}, SR_{A}, P(O)(OR_{A})₂, CO₂R_{A}, C(O)NR_{A}R_{B}, S(O)ₙR_{A}, S(O)NR_{A}R_{B}, or halogenated (e.g., fluorinated) alkyl, aryl or heteroaryl); and
n is 0-2.

2. The formulation of claim 1, wherein the compound is such that:
when R₁ is C(O)R_{A}, R₂ is CHF₂, and R₃ is 2,6-dichlorophenyl, R_{A} is not ethoxy, cyclopropyl, or isopropyl;
when R₁ is C(O)R_{A}, R₂ is H or CHF₂, and R₃ is 3,5-dimethylphenyl, R_{A} is not methoxy; or when R₁ is C(O)NR_{A}R_{B}, R₂ is pyrazyl, R₃ is 2,6-dimethyl-4-methoxyphenyl, and R_{A} is H, R_{B} is not ethyl.

3. The formulation of claim 1, wherein the compound is such that:
when R₁ is H, and R₂ is methyl, R₃ is not chloro.

4. The formulation of claim 2, wherein the compound is of the formula: 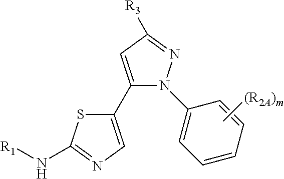
wherein each R_{2A} is independently cyano, halo, hydroxyl, NR_{A}R_{B}, SR_{A}, P(O)(OR_{A})₂, CO₂R_{A}, C(O)NR_{A}R_{B}, S(O)ₙR_{A}, S(O)NR_{A}R_{B}, or optionally substituted (e.g., optionally fluorinated) alkyl, alkoxyl, or aryl; and m is 0-5.

5. The formulation of claim 4, wherein the compound is of the formula:

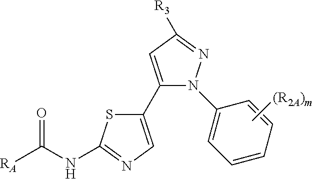

6. The formulation of claim 5, wherein R_{A} is alkyl optionally substituted with one or more of halo, hydroxyl, amino, alkylamino or dialkylamino.

7. The formulation of claim 6, wherein R_{A} is isopropyl.

8. The formulation of claim 6, wherein R_{A} is alkyl substituted with amino.

9. The formulation of claim 6, wherein at least one R_{2A} is chloro.

10. The formulation of claim 9, wherein the compound is of the formula:

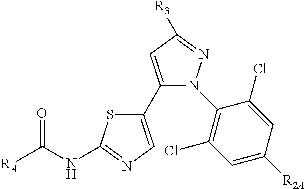

11. The formulation of claim 10, wherein at R_{2A} is bromo.

12. The formulation of claim 6, wherein m is 2 or 3.

13. The formulation of claim 6, wherein R₃ is H or optionally substituted lower alkyl.

14. The formulation of claim 13, wherein R₃ is difluoromethyl.

15. A compound of the formula: 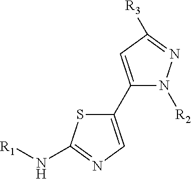
or a pharmaceutically acceptable salt thereof, wherein:
R₁ is H, C(O)R_{A}, S(O)ₙR_{A}, C(O)NR_{A}R_{B}, S(O)ₙNR_{A}R_{B}, S(O)ₙOR_{A}, C(NH)NR_{A}R_{B}, C(O)OR_{A}, C(S)NR_{A}R_{B}, C(SR_{B})NR_{A}, P(O)(OR_{A})₂or optionally substituted alkyl, aryl, or heterocycle (e.g., optionally substituted with halo, alkyl, alkoxyl, aryl, heteroaryl, hydroxyl, cyano, NR_{A}R_{B}, SR_{A}, P(O)(OR_{A})₂, CO₂R_{A}, C(O)NR_{A}R_{B}, S(O)ₙR_{A}, S(O)NR_{A}R_{B}, or halogenated (e.g., fluorinated) alkyl, aryl or heteroaryl);
R₂ is H, C(O)R_{A}, S(O)ₙR_{A}, C(O)NR_{A}R_{B}, S(O)ₙNR_{A}R_{B}, S(O)ₙOR_{A}, or optionally substituted alkyl, aryl, or heterocycle (e.g., optionally substituted with halo, alkyl, alkoxyl, aryl, heteroaryl, hydroxyl, cyano, NR_{A}R_{B}, SR_{A}, P(O)(OR_{A})₂, CO₂R_{A}, C(O)NR_{A}R_{B}, S(O)ₙR_{A}, S(O)NR_{A}R_{B}, or halogenated (e.g., fluorinated) alkyl, aryl or heteroaryl);
R₃ is H, halogen, OR, NR_{A}R_{B}, optionally substituted alkyl (e.g., optionally substituted with halo, alkyl, alkoxyl, hydroxyl, cyano, NR_{A}R_{B}, SR_{A}, CO₂R_{A}, C(O)NR_{A}R_{B};
each R_{A} is independently H or optionally substituted alkyl, aryl, alkylaryl, or alkyl-heterocycle (e.g., optionally substituted with halo, alkyl, alkoxyl, aryl, heteroaryl, hydroxyl, cyano, NR_{A}R_{B}, SR_{A}, P(O)(OR_{A})₂, CO₂R_{A}, C(O)NR_{A}R_{B}, S(O)ₙR_{A}, S(O)NR_{A}R_{B}, or halogenated (e.g., fluorinated) alkyl, aryl or heteroaryl);
each R_{B} is optionally substituted alkyl or aryl (e.g., optionally substituted with halo, alkyl, alkoxyl, aryl, heteroaryl, hydroxyl, cyano, NR_{A}R_{B}, SR_{A}, P(O)(OR_{A})₂, CO₂R_{A}, C(O)NR_{A}R_{B}, S(O)ₙR_{A}, S(O)NR_{A}R_{B}, or halogenated (e.g., fluorinated) alkyl, aryl or heteroaryl);
or when R_{A} and R_{B} are attached to the same nitrogen atom, they can be taken together with that nitrogen atom to form an optionally substituted heterocycle (e.g., piperidinyl, morpholino, thiomorpholino, piperazinyl, pyrrolidino, and azetidino optionally substituted with halo, alkyl, alkoxyl, aryl, heteroaryl, hydroxyl, cyano, NR_{A}R_{B}, SR_{A}, P(O)(OR_{A})₂, CO₂R_{A}, C(O)NR_{A}R_{B}, S(O)ₙR_{A}, S(O)NR_{A}R_{B}, or halogenated (e.g., fluorinated) alkyl, aryl or heteroaryl); and
n is 0-2;
with the provisos that:
when R₁ is C(O)R_{A}, R₂ is CHF₂, and R₃ is 2,6-dichlorophenyl, R_{A} is not ethoxy, cyclopropyl, or isopropyl;
when R₁ is C(O)R_{A}, R₂ is H or CHF₂, and R₃ is 3,5-dimethylphenyl, R_{A} is not methoxy;
when R₁ is C(O)NR_{A}R_{B}, R₂ is pyrazyl, R₃ is 2,6-dimethyl-4-methoxyphenyl, and R_{A} is H, R_{B} is not ethyl; and
when R₁ is H, and R₂ is methyl, R₃ is not chloro.

16. The compound of claim 15, which is of the formula: 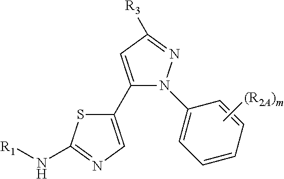
wherein each R_{2A} is independently cyano, halo, hydroxyl, NR_{A}R_{B}, SR_{A}, P(O)(OR_{A})₂, CO₂R_{A}, C(O)NR_{A}R_{B}, S(O)ₙR_{A}, S(O)NR_{A}R_{B}, or optionally substituted (e.g., optionally fluorinated) alkyl, alkoxyl, or aryl; and m is 0-5.

17. The compound of claim 16, which is of the formula:

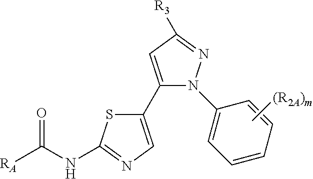

18. The compound of claim 17, which is of the formula:

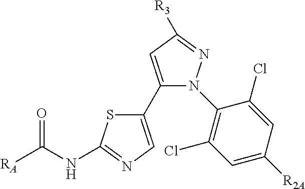

19. A method of lowering intraocular pressure in a patient, which comprises administering to a patient in need thereof a therapeutically or prophylactically effective amount of a compound of claim 1 or 15.

20. A method of treating, managing or preventing a disease or disorder affecting vision in a patient, which comprises administering to a patient in need thereof a therapeutically or prophylactically effective amount of a compound of claim 1 or 15.

21. The method of claim 20, wherein the disease or disorder affecting vision is glaucoma, neurodegeneration, or infection.
